(19) United States
(12) Patent Application Publication
(10) Pub. No.: US 2023/0034216 A1
(43) Pub. Date: Feb. 2, 2023

(54) MULTIPLEXED SPATIAL CAPTURE OF ANALYTES

(71) Applicant: 10x Genomics, Inc., Pleasanton, CA (US)

(72) Inventor: Felice Alessio Bava, Pleasanton, CA (US)

(21) Appl. No.: 17/873,788

(22) Filed: Jul. 26, 2022

Related U.S. Application Data
(60) Provisional application No. 63/226,460, filed on Jul. 28, 2021.

(51) Int. Cl. C12Q 1/6874; C12Q 1/6876; C12Q 1/6811; C12Q 1/6816

(52) U.S. Cl. CPC C12Q 1/6874; C12Q 1/6876; C12Q 1/6811; C12Q 1/6816

(57) ABSTRACT

Provided herein are methods, compositions, and kits for multiplex spatial detection of analytes in a biological sample.

### CROSS-REFERENCE TO RELATED APPLICATIONS

This application claims the benefit of priority to U.S. Provisional Application No. 63/226,460, filed on Jul. 28, 2021, the contents of which are hereby incorporated by reference in its entirety.

### BACKGROUND

Cells within a tissue have differences in cell morphology and/or function due to varied analyte levels (e.g., gene and/or protein expression) within the different cells. The specific position of a cell within a tissue (e.g., the cell's position relative to neighboring cells or the cell's position relative to the tissue microenvironment) can affect, e.g., the cell's morphology, differentiation, fate, viability, proliferation, behavior, signaling, and cross-talk with other cells in the tissue.

Spatial heterogeneity has been previously studied using techniques that typically provide data for a handful of analytes in the context of intact tissue or a portion of a tissue (e.g., tissue section), or provide significant analyte data from individual, single cells, but fails to provide information regarding the position of the single cells from the originating biological sample (e.g., tissue).

Methods for determining the presence of an analyte in a biological sample (e.g., nucleic acid, protein, etc.) are known in the art. Additionally, methods for determining the location of an analyte in a biological sample are known the art. However, multiplex methods for determining both the presence and/or abundance and the spatial location of multiple types of analytes (e.g., nucleic acid, protein, etc.) in a biological sample (e.g., a tissue section) are still needed.

### SUMMARY

A common challenge for multiplex methods for determining both the presence and spatial location of one or more types of analytes in a biological sample (e.g., a tissue section) results from the different experimental conditions required to capture different types of analytes. For example, conditions for optimal nucleic acid capture can be different than the conditions for optimal protein capture, particularly on an array. Thus, the present disclosure features methods, compositions, and kits for multiplex capture of analytes in a biological sample.

Provided herein are methods of determining a location of a first analyte and a location of a second analyte in a biological sample, the method including: (a) providing an array including a plurality of capture probes, where a first capture probe of the plurality of capture probes includes: (i) a first spatial barcode, and (ii) a first capture domain that hybridizes to a first analyte capture sequence; (b) contacting the biological sample with (i) a plurality of antigen-binding complexes, where an antigen-binding complex of the plurality of antigen-binding complexes includes: (1) an antigen-binding moiety that binds a first analyte, and (2) an oligonucleotide including an analyte-binding moiety barcode, where the analyte-binding moiety barcode identifies the antigen-binding complex; (ii) a plurality of capture oligonucleotides, where a capture oligonucleotide of the plurality of capture oligonucleotides includes: (1) a sequence substantially complementary to the analyte-binding moiety barcode, or a portion thereof, and (2) the first analyte capture sequence that hybridizes to the first capture domain of the first capture probe; (iii) a plurality of first probes, where a first probe of the plurality of first probes includes a sequence substantially complementary to a sequence of a second analyte and hybridizes to the second analyte, and where the first probe comprises a second analyte capture sequence that hybridizes to a second capture domain of a second capture probe; (c) determining (i) a sequence of the first spatial barcode, or a complement thereof, and (ii) the sequence of the analyte-binding moiety barcode, or a complement thereof, and correlating the determined sequences of (i) and (ii) to a location of the first analyte in the biological sample; and (d) determining (i) a sequence of the second spatial barcode, or a complement thereof, and (ii) the sequence of the first probe, or a complement thereof, and correlating the determined sequences of (i) and (ii) to a location of the second analyte in the biological sample.

In some embodiments, the method includes contacting a plurality of second probes with the biological sample, where the first probe and a second probe of the plurality of second probes each include one or more sequences substantially complementary to sequences of the second analyte and where the second probe hybridizes to the second analyte; and ligating the first probe and the second probe with a ligase, thereby generating a ligation product. In some embodiments, the method includes releasing the ligation product from the second analyte including the use of an RNase or heat.

In some embodiments, the ligation product including the second analyte capture sequence hybridizes to a second capture domain of a second capture probe, and where the second capture probe includes a second spatial barcode.

In some embodiments, the method includes determining (i) a sequence of a second spatial barcode, or a complement thereof, and (ii) the sequence of the ligation product, or a complement thereof, and correlating the determined sequences of (i) and (ii) to a location of the second analyte in the biological sample.

In some embodiments, the first antigen-binding complex is an antibody, or an antigen-binding fragment thereof, and the first analyte includes a protein and the second analyte includes a nucleic acid.

In some embodiments, after step (b) the method includes removing one or more unbound antigen-binding complexes and/or removing one or more unbound first probes or one or more unbound second probes.

In some embodiments, the first capture domain and the second capture domain include a poly(T) sequence or wherein the first capture domain and the second capture domain include different sequences.

In some embodiments, the first capture probe and the second capture probe include: one or more functional domains, a unique molecule identifier, a cleavage domain, and combinations thereof.

In some embodiments, the method includes extending the first capture probe using the capture oligonucleotide as a template and/or extending the second capture probe as a template using the first probe as a template, where the extending includes the use of a polymerase.

In some embodiments, the method includes extending the second capture probe using the ligation product as a template, where the extending includes the use of a polymerase.

In some embodiments, the method includes permeabilizing the biological sample.

In some embodiments, the method includes imaging the biological sample and/or staining the biological sample, where the staining comprises hematoxylin and eosin staining or immunofluorescence.

In some embodiments, the biological sample is a tissue sample or a tissue section, where the tissue section is a fresh-frozen tissue section or a fixed tissue section, wherein the fixed tissue section comprises a formalin-fixed paraffin-embedded tissue section, a paraformaldehyde-fixed tissue section, an acetone-fixed tissue section, or a methanol-fixed tissue section.

In some embodiments, the determining in step (e), step (f), or both includes sequencing.

In some embodiments, the antigen-binding complex includes a linker, where the linker is disposed between the analyte-binding moiety and the analyte-binding moiety barcode where the linker is a cleavable linker, and where the cleavable linker is a photo-cleavable linker or an enzyme cleavable linker.

In some embodiments, the method includes blocking the first analyte capture sequence and/or second analyte capture sequence with one or more blocking probes.

In some embodiments, the biological sample is disposed on the array. In some embodiments, the biological sample is disposed on a substrate. In some embodiments, the method includes aligning the substrate with the array, such that at least a portion of the biological sample is aligned with at least a portion of the array.

All publications, patents, and patent applications mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

The term “each,” when used in reference to a collection of items, is intended to identify an individual item in the collection but does not necessarily refer to every item in the collection, unless expressly stated otherwise, or unless the context of the usage clearly indicates otherwise.

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

### DESCRIPTION OF DRAWINGS

The following drawings illustrate certain embodiments of the features and advantages of this disclosure. These embodiments are not intended to limit the scope of the appended claims in any manner. Like reference symbols in the drawings indicate like elements.

FIG. 1 is a schematic diagram showing an example of a barcoded capture probe, as described herein.

FIG. 2 is a schematic diagram of an exemplary antigen-binding complex.

FIG. 3 is an exemplary workflow showing multiplex capture of analytes from a biological sample.

FIG. 4 is a diagram showing capture of analyte proxies on an arrayed slide.

FIG. 5 is a schematic diagram depicting an exemplary sandwiching process between a first substrate comprising a biological sample and a second substrate comprising a spatially barcoded array.

FIGS. 6A-B are schematic diagrams depicting exemplary sandwiching embodiments. FIG. 6A shows an exemplary sandwiching process where a first substrate including a biological sample and a second substrate are brought into proximity with one another and a liquid reagent drop is introduced on the second substrate in proximity to the capture probes and in between the biological sample. FIG. 6B shows a fully formed sandwich configuration creating a chamber formed from one or more spacers, the first substrate, and the second substrate including spatially barcoded capture probes.

### DETAILED DESCRIPTION

A common challenge for multiplex methods for determining both the presence and spatial location of one or more types of analytes in a biological sample (e.g., a tissue section) results from the different experimental conditions required to capture different types of analytes. For example, conditions for optimal nucleic acid capture can be different than the conditions for optimal protein capture, particularly on an array. In some embodiments, the biological sample is disposed on the array. In some embodiments, the biological sample is disposed on a substrate and then aligned with the array (e.g., “sandwiched”). Thus, in some examples, a first analyte is captured under a first set of conditions, followed by a second set of conditions to capture a second analyte. For example, a protein can be captured by an antibody or antigen-binding fragment thereof, where the antibody or antigen-binding fragment is conjugated to an oligonucleotide. In some embodiments, the oligonucleotide includes an analyte-binding moiety barcode. In some embodiments, the oligonucleotide comprises a sequence complementary to a capture oligonucleotide, or a portion thereof. In some examples, experimental conditions for ideal protein capture are maintained followed by capture for a different analyte, such as nucleic acid. For example, reagents for nucleic acid (e.g., mRNA) capture can be delivered to the sample under a different set of conditions than protein capture. In some embodiments, one probe is delivered to the sample. In some embodiments, two probes (e.g., a first and a second probe) are delivered to the biological sample. In some embodiments, the probe (or a first and a second probe) can hybridize to the nucleic acid. In some embodiments, the first probe includes a sequence complementary to the capture domain of a capture probe (e.g., an analyte capture sequence). Thus, the conditions for nucleic acid capture can facilitate capture of the capture oligonucleotide and/or the probe (e.g., including either the first or second probe that includes the analyte capture sequence) on the array resulting in multiplex capture of two different types of analytes.

The present disclosure features methods, compositions, and kits for multiplex capture of analytes in a biological sample.

Spatial analysis methodologies and compositions described herein can provide a vast amount of analyte and/or expression data for a variety of analytes within a biological sample at high spatial resolution, while retaining native spatial context. Spatial analysis methods and compositions can include, e.g., the use of a capture probe including a spatial barcode (e.g., a nucleic acid sequence that provides information as to the location or position of an analyte within a cell or a tissue sample (e.g., mammalian cell or a mammalian tissue sample) and a capture domain that is capable of binding to an analyte (e.g., a protein and/or a nucleic acid) produced by and/or present in a cell. Spatial analysis methods and compositions can also include the use of a capture probe having a capture domain that captures an intermediate agent for indirect detection of an analyte. For example, the intermediate agent can include a nucleic acid sequence (e.g., a barcode) associated with the intermediate agent. Detection of the intermediate agent is therefore indicative of the analyte in the cell or tissue sample.

Non-limiting aspects of spatial analysis methodologies and compositions are described in U.S. Pat. Nos. 10,774,374, 10,724,078, 10,480,022, 10,059,990, 10,041,949, 10,002,316, 9,879,313, 9,783,841, 9,727,810, 9,593,365, 8,951,726, 8,604,182, 7,709,198, U.S. Patent Application Publication Nos. 2020/239946, 2020/080136, 2020/0277663, 2020/024641, 2019/330617, 2019/264268, 2020/256867, 2020/224244, 2019/194709, 2019/161796, 2019/085383, 2019/055594, 2018/216161, 2018/051322, 2018/0245142, 2017/241911, 2017/089811, 2017/067096, 2017/029875, 2017/0016053, 2016/108458, 2015/000854, 2013/171621, WO 2018/091676, WO 2020/176788, Rodrigues et al., Science 363(6434):1463-1467, 2019; Lee et al., Nat. Protoc. 10(3):442-458, 2015; Trejo et al., PLoS ONE 14(2):e0212031, 2019; Chen et al., Science 348(6233):aaa6090, 2015; Gao et al., BMC Biol. 15:50, 2017; and Gupta et al., Nature Biotechnol. 36:1197-1202, 2018; the Visium Spatial Gene Expression Reagent Kits User Guide (e.g., Rev C, dated June 2020), and/or the Visium Spatial Tissue Optimization Reagent Kits User Guide (e.g., Rev C, dated July 2020), both of which are available at the 10× Genomics Support Documentation website, and can be used herein in any combination. Further non-limiting aspects of spatial analysis methodologies and compositions are described herein.

Some general terminology that may be used in this disclosure can be found in Section (I)(b) of WO 2020/176788 and/or U.S. Patent Application Publication No. 2020/0277663. Typically, a “barcode” is a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes. For the purpose of this disclosure, an “analyte” can include any biological substance, structure, moiety, or component to be analyzed. The term “target” can similarly refer to an analyte of interest.

Analytes can be broadly classified into one of two groups: nucleic acid analytes, and non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. In some embodiments, the analyte(s) can be localized to subcellular location(s), including, for example, organelles, e.g., mitochondria, Golgi apparatus, endoplasmic reticulum, chloroplasts, endocytic vesicles, exocytic vesicles, vacuoles, lysosomes, etc. In some embodiments, analyte(s) can be peptides or proteins, including without limitation antibodies and enzymes. Additional examples of analytes can be found in Section (I)(c) of WO 2020/176788 and/or U.S. Patent Application Publication No. 2020/0277663. In some embodiments, an analyte can be detected indirectly, such as through detection of an intermediate agent, for example, a ligation product or an analyte capture agent (e.g., an oligonucleotide-conjugated antibody), such as those described herein.

A “biological sample” is typically obtained from the subject for analysis using any of a variety of techniques including, but not limited to, biopsy, surgery, and laser capture microscopy (LCM), and generally includes cells and/or other biological material from the subject. In some embodiments, a biological sample can be a tissue section. In some embodiments, a biological sample can be a fixed and/or stained biological sample (e.g., a fixed and/or stained tissue section). Non-limiting examples of stains include histological stains (e.g., hematoxylin and/or eosin) and immunological stains (e.g., fluorescent stains). In some embodiments, a biological sample (e.g., a fixed and/or stained biological sample) can be imaged. Biological samples are also described in Section (I)(d) of WO 2020/176788 and/or U.S. Patent Application Publication No. 2020/0277663.

In some embodiments, a biological sample is permeabilized with one or more permeabilization reagents. For example, permeabilization of a biological sample can facilitate analyte capture. Exemplary permeabilization agents and conditions are described in Section (I)(d)(ii)(13) or the Exemplary Embodiments Section of WO 2020/176788 and/or U.S. Patent Application Publication No. 2020/0277663.

Array-based spatial analysis methods involve the transfer of one or more analytes from a biological sample to an array of features on a substrate, where each feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array.

A “capture probe” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe includes a barcode (e.g., a spatial barcode and/or a unique molecular identifier (UMI)) and a capture domain). In some embodiments, a capture probe can include a cleavage domain and/or a functional domain (e.g., a primer-binding site, such as for next-generation sequencing (NGS)). See, e.g., Section (II)(b) (e.g., subsections (i)-(vi)) of WO 2020/176788 and/or U.S. Patent Application Publication No. 2020/0277663. Generation of capture probes can be achieved by any appropriate method, including those described in Section (II)(d)(ii) of WO 2020/176788 and/or U.S. Patent Application Publication No. 2020/0277663.

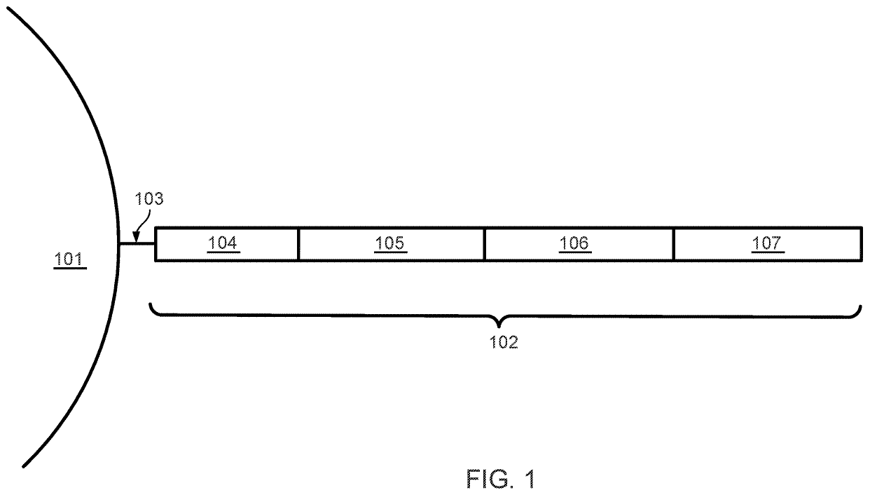

FIG. 1 is a schematic diagram showing an exemplary capture probe, as described herein. As shown, the capture probe 102 is optionally coupled to a feature 101 by a cleavage domain 103, such as a disulfide linker. The capture probe can include a functional sequence 104 that is useful for subsequent processing. The functional sequence 104 can include all or a part of sequencer specific flow cell attachment sequence (e.g., a P5 or P7 sequence), all or a part of a sequencing primer sequence, (e.g., a R1 primer binding site, a R2 primer binding site), or combinations thereof. The capture probe can also include a spatial barcode 105. The capture probe can also include a unique molecular identifier (UMI) sequence 106. While FIG. 1 shows the spatial barcode 105 as being located upstream (5′) of UMI sequence 106, it is to be understood that capture probes wherein UMI sequence 106 is located upstream (5′) of the spatial barcode 105 is also suitable for use in any of the methods described herein. The capture probe can also include a capture domain 107 to facilitate capture of a target analyte. In some embodiments, the capture probe comprises one or more additional functional sequences that can be located, for example between the spatial barcode 105 and the UMI sequence 106, between the UMI sequence 106 and the capture domain 107, or following the capture domain 107. The capture domain can have a sequence complementary to a sequence of a nucleic acid analyte. The capture domain can have a sequence complementary to a capture handle sequence present in an analyte capture agent. The capture domain can have a sequence complementary to a splint oligonucleotide. Such splint oligonucleotide, in addition to having a sequence complementary to a capture domain of a capture probe, can have a sequence of a nucleic acid analyte, and/or a capture handle sequence described herein.

The functional sequences can generally be selected for compatibility with any of a variety of different sequencing systems, e.g., Ion Torrent Proton or PGM, Illumina sequencing instruments, PacBio, Oxford Nanopore, etc., and the requirements thereof. In some embodiments, functional sequences can be selected for compatibility with non-commercialized sequencing systems. Examples of such sequencing systems and techniques, for which suitable functional sequences can be used, include (but are not limited to) Ion Torrent Proton or PGM sequencing, Illumina sequencing, PacBio SMRT sequencing, and Oxford Nanopore sequencing. Further, in some embodiments, functional sequences can be selected for compatibility with other sequencing systems, including non-commercialized sequencing systems.

In some embodiments, the spatial barcode 105 and functional sequences 104 is common to all of the probes attached to a given feature (e.g., a bead, a well, a spot on an array). In some embodiments, the UMI sequence 106 of a capture probe attached to a given feature is different from the UMI sequence of a different capture probe attached to the given feature.

There are at least two methods to associate a spatial barcode with one or more neighboring cells, such that the spatial barcode identifies the one or more cells, and/or contents of the one or more cells, as associated with a particular spatial location. One method is to promote analytes or analyte proxies (e.g., intermediate agents) out of a cell and towards a spatially-barcoded array (e.g., including spatially-barcoded capture probes). Another method is to cleave spatially-barcoded capture probes from an array and promote the spatially-barcoded capture probes towards and/or into or onto the biological sample.

In some cases, capture probes may be configured to prime, replicate, and consequently yield optionally barcoded extension products from a template (e.g., a DNA or RNA template, such as an analyte or an intermediate agent (e.g., an extension product, a ligation product or an analyte capture agent), or a portion thereof), or derivatives thereof (see, e.g., Section (II)(b)(vii) of WO 2020/176788 and/or U.S. Patent Application Publication No. 2020/0277663 regarding extended capture probes). In some cases, capture probes may be configured to form ligation products with a template (e.g., a DNA or RNA template, such as an analyte or an intermediate agent, or portion thereof), thereby creating ligations products that serve as proxies for a template.

In some embodiments, more than one analyte type (e.g., nucleic acids and proteins) from a biological sample can be detected (e.g., simultaneously or sequentially) using any appropriate multiplexing technique, such as those described in Section (IV) of WO 2020/176788 and/or U.S. Patent Application Publication No. 2020/0277663.

There are at least two methods to associate a spatial barcode with one or more neighboring cells, such that the spatial barcode identifies the one or more cells, and/or contents of the one or more cells, as associated with a particular spatial location. One method is to promote analytes or analyte proxies (e.g., intermediate agents) out of a cell and towards a spatially-barcoded array (e.g., including spatially-barcoded capture probes). In some examples, the biological sample is disposed on the spatially-barcoded array. In some examples, the biological sample is on a substrate and aligned with the spatially-barcoded array Another method is to cleave spatially-barcoded capture probes from an array and promote the spatially-barcoded capture probes towards and/or into or onto the biological sample.

In some cases, capture probes may be configured to prime, replicate, and consequently yield optionally barcoded extension products from a template (e.g., a DNA or RNA template, such as an analyte or an intermediate agent (e.g., a ligation product or an analyte capture agent), or a portion thereof), or derivatives thereof (see, e.g., Section (II)(b)(vii) of WO 2020/176788 and/or U.S. Patent Application Publication No. 2020/0277663 regarding extended capture probes). In some cases, capture probes may be configured to form ligation products with a template (e.g., a DNA or RNA template, such as an analyte or an intermediate agent, or portion thereof), thereby creating ligations products that serve as proxies for a template.

As used herein, an “extended capture probe” refers to a capture probe having additional nucleotides added to the terminus (e.g., 3′ or 5′ end) of the capture probe thereby extending the overall length of the capture probe. For example, an “extended 3′ end” indicates additional nucleotides were added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some embodiments, extending the capture probe includes adding to a 3′ end of a capture probe a nucleic acid sequence that is complementary to a nucleic acid sequence of an analyte or intermediate agent specifically bound to the capture domain of the capture probe. In some embodiments, the capture probe is extended using reverse transcription. In some embodiments, the capture probe is extended using one or more DNA polymerases. The extended capture probes include the sequence of the capture probe and the sequence of the spatial barcode of the capture probe.

In some embodiments, extended capture probes are amplified (e.g., in bulk solution or on the array) to yield quantities that are sufficient for downstream analysis, e.g., via DNA sequencing. In some embodiments, extended capture probes (e.g., DNA molecules) act as templates for an amplification reaction (e.g., a polymerase chain reaction).

Additional variants of spatial analysis methods, including in some embodiments, an imaging step, are described in Section (II)(a) of WO 2020/176788 and/or U.S. Patent Application Publication No. 2020/0277663. Analysis of captured analytes (and/or intermediate agents or portions thereof), for example, including sample removal, extension of capture probes, sequencing (e.g., of a cleaved extended capture probe and/or a cDNA molecule complementary to an extended capture probe), sequencing on the array (e.g., using, for example, in situ hybridization or in situ ligation approaches), temporal analysis, and/or proximity capture, is described in Section (II)(g) of WO 2020/176788 and/or U.S. Patent Application Publication No. 2020/0277663. Some quality control measures are described in Section (II)(h) of WO 2020/176788 and/or U.S. Patent Application Publication No. 2020/0277663.

Spatial information can provide information of biological and/or medical importance. For example, the methods and compositions described herein can allow for: identification of one or more biomarkers (e.g., diagnostic, prognostic, and/or for determination of efficacy of a treatment) of a disease or disorder; identification of a candidate drug target for treatment of a disease or disorder; identification (e.g., diagnosis) of a subject as having a disease or disorder; identification of stage and/or prognosis of a disease or disorder in a subject; identification of a subject as having an increased likelihood of developing a disease or disorder; monitoring of progression of a disease or disorder in a subject; determination of efficacy of a treatment of a disease or disorder in a subject; identification of a patient subpopulation for which a treatment is effective for a disease or disorder; modification of a treatment of a subject with a disease or disorder; selection of a subject for participation in a clinical trial; and/or selection of a treatment for a subject with a disease or disorder.

Spatial information can provide information of biological importance. For example, the methods and compositions described herein can allow for: identification of transcriptome and/or proteome expression profiles (e.g., in healthy and/or diseased tissue); identification of multiple analyte types in close proximity (e.g., nearest neighbor analysis); determination of up- and/or down-regulated genes and/or proteins in diseased tissue; characterization of tumor microenvironments; characterization of tumor immune responses; characterization of cells types and their co-localization in tissue; and identification of genetic variants within tissues (e.g., based on gene and/or protein expression profiles associated with specific disease or disorder biomarkers).

Typically, for spatial array-based methods, a substrate functions as a support for direct or indirect attachment of capture probes to features of the array. A “feature” is an entity that acts as a support or repository for various molecular entities used in spatial analysis. In some embodiments, some or all of the features in an array are functionalized for analyte capture. Exemplary substrates are described in Section (II)(c) of WO 2020/176788 and/or U.S. Patent Application Publication No. 2020/0277663. Exemplary features and geometric attributes of an array can be found in Sections (II)(d)(i), (II)(d)(iii), and (II)(d)(iv) of WO 2020/176788 and/or U.S. Patent Application Publication No. 2020/0277663.

Generally, analytes and/or intermediate agents (or portions thereof) can be captured when contacting a biological sample with a substrate including capture probes (e.g., a substrate with capture probes embedded, spotted, printed, fabricated on the substrate, or a substrate with features (e.g., beads, wells) comprising capture probes). As used herein, “contact,” “contacted,” and/or “contacting,” a biological sample with a substrate refers to any contact (e.g., direct or indirect) such that capture probes can interact (e.g., bind covalently or non-covalently (e.g., hybridize)) with analytes from the biological sample. Capture can be achieved actively (e.g., using electrophoresis) or passively (e.g., using diffusion). Analyte capture is further described in Section (II)(e) of WO 2020/176788 and/or U.S. Patent Application Publication No. 2020/0277663.

In some cases, spatial analysis can be performed by attaching and/or introducing a molecule (e.g., a peptide, a lipid, or a nucleic acid molecule) having a barcode (e.g., a spatial barcode) to a biological sample (e.g., to a cell in a biological sample). In some embodiments, a plurality of molecules (e.g., a plurality of nucleic acid molecules) having a plurality of barcodes (e.g., a plurality of spatial barcodes) are introduced to a biological sample (e.g., to a plurality of cells in a biological sample) for use in spatial analysis. In some embodiments, after attaching and/or introducing a molecule having a barcode to a biological sample, the biological sample can be physically separated (e.g., dissociated) into single cells or cell groups for analysis. Some such methods of spatial analysis are described in Section (III) of WO 2020/176788 and/or U.S. Patent Application Publication No. 2020/0277663.

In some cases, spatial analysis can be performed by detecting multiple oligonucleotides that hybridize to an analyte. In some instances, for example, spatial analysis can be performed using RNA-templated ligation (RTL). Methods of RTL have been described previously. See, e.g., Credle et al., Nucleic Acids Res. 2017 Aug. 21; 45(14):e128. Typically, RTL includes hybridization of two oligonucleotides to adjacent sequences on an analyte (e.g., an RNA molecule, such as an mRNA molecule). In some instances, the oligonucleotides are DNA molecules. In some instances, one of the oligonucleotides includes at least two ribonucleic acid bases at the 3′ end and/or the other oligonucleotide includes a phosphorylated nucleotide at the 5′ end. In some instances, one of the two oligonucleotides includes a capture domain (e.g., a poly(A) sequence, a non-homopolymeric sequence). After hybridization to the analyte, a ligase (e.g., SplintR ligase) ligates the two oligonucleotides together, creating a ligation product. In some instances, the two oligonucleotides hybridize to sequences that are not adjacent to one another. For example, hybridization of the two oligonucleotides creates a gap between the hybridized oligonucleotides. In some instances, a polymerase (e.g., a DNA polymerase) can extend one of the oligonucleotides prior to ligation. After ligation, the ligation product is released from the analyte. In some instances, the ligation product is released using an endonuclease (e.g., RNAse H). The released ligation product can then be captured by capture probes (e.g., instead of direct capture of an analyte) on an array, optionally amplified, and sequenced, thus determining the location and optionally the abundance of the analyte in the biological sample.

During analysis of spatial information, sequence information for a spatial barcode associated with an analyte is obtained, and the sequence information can be used to provide information about the spatial distribution of the analyte in the biological sample. Various methods can be used to obtain the spatial information. In some embodiments, specific capture probes and the analytes they capture are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Alternatively, specific spatial barcodes can be deposited at predetermined locations in an array of features during fabrication such that at each location, only one type of spatial barcode is present so that spatial barcodes are uniquely associated with a single feature of the array. Where necessary, the arrays can be decoded using any of the methods described herein so that spatial barcodes are uniquely associated with array feature locations, and this mapping can be stored as described above.

When sequence information is obtained for capture probes and/or analytes during analysis of spatial information, the locations of the capture probes and/or analytes can be determined by referring to the stored information that uniquely associates each spatial barcode with an array feature location. In this manner, specific capture probes and captured analytes are associated with specific locations in the array of features. Each array feature location represents a position relative to a coordinate reference point (e.g., an array location, a fiducial marker) for the array. Accordingly, each feature location has an “address” or location in the coordinate space of the array.

Some exemplary spatial analysis workflows are described in the Exemplary Embodiments section of WO 2020/176788 and/or U.S. Patent Application Publication No. 2020/0277663. See, for example, the Exemplary embodiment starting with “In some non-limiting examples of the workflows described herein, the sample can be immersed . . . ” of WO 2020/176788 and/or U.S. Patent Application Publication No. 2020/0277663. See also, e.g., the Visium Spatial Gene Expression Reagent Kits User Guide (e.g., Rev C, dated June 2020), and/or the Visium Spatial Tissue Optimization Reagent Kits User Guide (e.g., Rev C, dated July 2020).

In some embodiments, spatial analysis can be performed using dedicated hardware and/or software, such as any of the systems described in Sections (II)(e)(ii) and/or (V) of WO 2020/176788 and/or U.S. Patent Application Publication No. 2020/0277663, or any of one or more of the devices or methods described in Sections Control Slide for Imaging, Methods of Using Control Slides and Substrates for, Systems of Using Control Slides and Substrates for Imaging, and/or Sample and Array Alignment Devices and Methods, Informational labels of WO 2020/123320.

Suitable systems for performing spatial analysis can include components such as a chamber (e.g., a flow cell or sealable, fluid-tight chamber) for containing a biological sample. The biological sample can be mounted for example, in a biological sample holder. One or more fluid chambers can be connected to the chamber and/or the sample holder via fluid conduits, and fluids can be delivered into the chamber and/or sample holder via fluidic pumps, vacuum sources, or other devices coupled to the fluid conduits that create a pressure gradient to drive fluid flow. One or more valves can also be connected to fluid conduits to regulate the flow of reagents from reservoirs to the chamber and/or sample holder.

The systems can optionally include a control unit that includes one or more electronic processors, an input interface, an output interface (such as a display), and a storage unit (e.g., a solid state storage medium such as, but not limited to, a magnetic, optical, or other solid state, persistent, writeable and/or re-writeable storage medium). The control unit can optionally be connected to one or more remote devices via a network. The control unit (and components thereof) can generally perform any of the steps and functions described herein. Where the system is connected to a remote device, the remote device (or devices) can perform any of the steps or features described herein. The systems can optionally include one or more detectors (e.g., CCD, CMOS) used to capture images. The systems can also optionally include one or more light sources (e.g., LED-based, diode-based, lasers) for illuminating a sample, a substrate with features, analytes from a biological sample captured on a substrate, and various control and calibration media.

The systems can optionally include software instructions encoded and/or implemented in one or more of tangible storage media and hardware components such as application specific integrated circuits. The software instructions, when executed by a control unit (and in particular, an electronic processor) or an integrated circuit, can cause the control unit, integrated circuit, or other component executing the software instructions to perform any of the method steps or functions described herein.

In some cases, the systems described herein can detect (e.g., register an image) the biological sample on the array. Exemplary methods to detect the biological sample on an array are described in PCT Application No. 2020/061064 and/or U.S. patent application Ser. No. 16/951,854.

Prior to transferring analytes from the biological sample to the array of features on the substrate, the biological sample can be aligned with the array. Alignment of a biological sample and an array of features including capture probes can facilitate spatial analysis, which can be used to detect differences in analyte presence and/or level within different positions in the biological sample, for example, to generate a three-dimensional map of the analyte presence and/or level. Exemplary methods to generate a two- and/or three-dimensional map of the analyte presence and/or level are described in PCT Application No. 2020/053655 and spatial analysis methods are generally described in WO 2020/061108 and/or U.S. patent application Ser. No. 16/951,864.

In some cases, a map of analyte presence and/or level can be aligned to an image of a biological sample using one or more fiducial markers, e.g., objects placed in the field of view of an imaging system which appear in the image produced, as described in the Substrate Attributes Section, Control Slide for Imaging Section of WO 2020/123320, PCT Application No. 2020/061066, and/or U.S. patent application Ser. No. 16/951,843. Fiducial markers can be used as a point of reference or measurement scale for alignment (e.g., to align a sample and an array, to align two substrates, to determine a location of a sample or array on a substrate relative to a fiducial marker) and/or for quantitative measurements of sizes and/or distances.

### Multiplex Detection of Analytes in a Biological Sample

Methods for determining the presence of an analyte in a biological sample (e.g., nucleic acid, protein, etc.) are known in the art. Additionally, methods for determining the location of an analyte in a biological sample are known the art. However, multiplex methods for determining both the presence and spatial location of one or more types of analytes in a biological sample (e.g., nucleic acid, protein, etc.) are still needed. A common challenge for multiplex methods for determining both the presence and spatial location of one or more types of analytes in a biological sample (e.g., a tissue section) results from the different experimental conditions required to capture different types of analytes. For example, conditions for optimal nucleic acid capture can be different than the conditions for optimal protein capture. Thus, in some examples, a first analyte is captured under a first set of conditions, followed by a second set of conditions to capture a second analyte. For example, a protein can be captured by an antibody or antigen-binding fragment thereof, where the antibody or antigen-binding fragment is conjugated to an oligonucleotide (e.g., an antigen-binding complex). In some embodiments, the oligonucleotide comprises a sequence complementary to a capture domain of a capture probe (e.g., an analyte capture sequence). In some examples, after the first analyte is bound (e.g., protein), the conditions can be altered for capture of a different analyte, such as nucleic acid. For example, reagents for nucleic acid (e.g., mRNA) capture can be delivered to the biological sample under a different set of conditions than protein capture. In some embodiments, one probe is delivered to the biological sample. In some embodiments, two probes (e.g., a first and a second probe) are delivered to the biological sample. In some embodiments, the probe (or a first and a second probe) can hybridize to the target nucleic acid. In some embodiments, the probe includes a sequence complementary to the capture domain of a capture probe (e.g., a second analyte capture sequence). Thus, the conditions for nucleic acid capture can facilitate capture of the capture oligonucleotide and/or the probe (e.g., including either the first or second probe that includes the analyte capture sequence) on the array resulting in multiplex capture of two different types of analytes. In some embodiments, the biological sample is disposed on the array including the plurality of capture probes. In some embodiments, the biological sample is disposed on a substrate and aligned with the array including a plurality of capture probes (e.g., sandwiched).

Thus, provided herein are methods of determining a location of a first analyte and a location of a second analyte in a biological sample, the method including: a) providing an array including a plurality of capture probes, where a first capture probe of the plurality of capture probes includes: (i) a first spatial barcode, and (ii) a first capture domain that hybridizes to a first analyte capture sequence; b) contacting the biological sample with a plurality of antigen-binding complexes, where an antigen-binding complex of the plurality of antigen-binding complexes includes: (i) an antigen-binding moiety that binds a first analyte, and (ii) an oligonucleotide including an analyte-binding moiety barcode, where the analyte-binding moiety barcode identifies the antigen-binding complex; c) contacting the biological sample with a plurality of capture oligonucleotides, where a capture oligonucleotide of the plurality of capture oligonucleotides includes: (i) a sequence substantially complementary to the analyte-binding moiety barcode, or a portion thereof, and (ii) the first analyte capture sequence; d) contacting a plurality of first probes with the biological sample, wherein a first probe of the plurality of first probes comprises a sequence substantially complementary to a sequence of a second analyte and hybridizes to the second analyte, and wherein the first probe comprises a second analyte capture sequence that hybridizes to a second capture domain of a second capture probe; e) determining (i) a sequence of the first spatial barcode, or a complement thereof, and (ii) all or a portion of the sequence of the analyte-binding moiety barcode, or a complement thereof, and correlating the determined sequences of (i) and (ii) to a location of the first analyte in the biological sample; and f) determining (i) a sequence of the second spatial barcode, or a complement thereof, and (ii) all or a portion of the sequence of the first probe, or a complement thereof, and correlating the determined sequences of (i) and (ii) to a location of the second analyte in the biological sample.

In some embodiments, steps (c) and (d) are performed simultaneously. In some embodiments, steps (c) and (d) are performed sequentially. In some embodiments, steps (b), (c), and (d) are performed simultaneously. In some embodiments, steps (b), (c), and (d) are performed sequentially.

Also provided herein are methods of determining a location of a first analyte and a location of a second analyte in a biological sample, the method including: (a) providing an array including a plurality of capture probes, where a first capture probe of the plurality of capture probes includes: (i) a first spatial barcode, and (ii) a first capture domain that hybridizes to a first analyte capture sequence; (b) contacting the biological sample with (i) a plurality of antigen-binding complexes, where an antigen-binding complex of the plurality of antigen-binding complexes includes: (1) an antigen-binding moiety that binds a first analyte, and (2) an oligonucleotide including an analyte-binding moiety barcode, where the analyte-binding moiety barcode identifies the antigen-binding complex; (ii) a plurality of capture oligonucleotides, where a capture oligonucleotide of the plurality of capture oligonucleotides includes: (1) a sequence substantially complementary to the analyte-binding moiety barcode, or a portion thereof, and (2) the first analyte capture sequence that hybridizes to the first capture domain of the first capture probe; (iii) a plurality of first probes, where a first probe of the plurality of first probes includes a sequence substantially complementary to a sequence of a second analyte and hybridizes to the second analyte, and where the first probe comprises a second analyte capture sequence that hybridizes to a second capture domain of a second capture probe; (c) determining (i) a sequence of the first spatial barcode, or a complement thereof, and (ii) the sequence of the analyte-binding moiety barcode, or a complement thereof, and correlating the determined sequences of (i) and (ii) to a location of the first analyte in the biological sample; and (d) determining (i) a sequence of the second spatial barcode, or a complement thereof, and (ii) the sequence of the first probe, or a complement thereof, and correlating the determined sequences of (i) and (ii) to a location of the second analyte in the biological sample.

In some embodiments, the method includes contacting a plurality of second probes with the biological sample, where the first probe and a second probe of the plurality of second probes each include one or more sequences substantially complementary to sequences of the second analyte and wherein the second probe hybridizes to the second analyte.

As used herein, “capture oligonucleotide” refers to an oligonucleotide that is substantially complementary to a least a portion of an oligonucleotide conjugated to an antigen-binding complex. The capture oligonucleotide can be complementary to the analyte-binding moiety barcode (as described herein) sequence included in the oligonucleotide conjugated to the antigen-binding complex and/or adjacent sequences. The capture oligonucleotide also includes an analyte capture sequence (e.g., a first analyte capture sequence), as described herein, capable of binding (e.g., hybridizing) to a capture domain of a capture probe (e.g., a first capture probe).

In some embodiments, the capture oligonucleotide can differ in length and/or complexity. In some embodiments, the capture oligonucleotide can include a nucleotide sequence of about 8 to about 50 nucleotides in length (e.g., about 8 to about 48, about 8 to about 46, about 8 to about 44, about 8 to about 42, about 8 to about 40, about 8 to about 38, about 8 to about 36, about 10 to about 50, about 10 to about 48, about 10 to about 46, about 10 to about 44, about 10 to about 42, about 10 to about 40, about 10 to about 38, about 12 to about 50, about 12 to about 48, about 12 to about 46, about 12 to about 44, about 12 to about 42, about 12 to about 40, about 14 to about 50, about 14 to about 48, about 14 to about 46, about 14 to about 44, about 14 to about 42, about 16 to about 50, about 16 to about 48, about 16 to about 44, about 16 to about 42, about 18 to about 40, about 18 to about 50, about 18 to about 48, about 20 to about 50, about 20 to about 48, or about 22 to about 48 nucleotides in length).

As used herein, an “antigen-binding complex” refers to a molecule that interacts with an analyte (e.g., a protein in a biological sample) and with a capture oligonucleotide to identify the analyte. In some embodiments, the antigen-binding complex includes: (i) an analyte binding moiety (e.g., a moiety that binds to an analyte), for example, an antibody or antigen-binding fragment thereof and (ii) an analyte-binding moiety barcode. As described herein, the term “analyte-binding moiety barcode” refers to a barcode that is associated with or otherwise identifies the analyte binding moiety.

In some embodiments, the conjugated oligonucleotide coupled to the analyte binding moiety includes a cleavable domain. For example, after the antigen-binding complex binds to an analyte and the capture oligonucleotide binds (e.g., hybridizes) to the conjugated oligonucleotide, the conjugated oligonucleotide can be cleaved and collected for downstream analysis according to the methods as described herein. In some embodiments, the cleavable domain of the conjugated oligonucleotide includes a uracil or U-excising element. In some embodiments, the U-excising element can include a single-stranded DNA (ssDNA) sequence that contains at least one uracil. The species can be attached to a bead via the ssDNA sequence. The species can be released by a combination of uracil-DNA glycosylase (e.g., to remove the uracil) and an endonuclease (e.g., to induce an ssDNA break). If the endonuclease generates a 5′ phosphate group from the cleavage, then additional enzyme treatment can be included in downstream processing to eliminate the phosphate group, e.g., prior to ligation of additional sequencing handle elements, e.g., Illumina full P5 sequence, partial P5 sequence, full R1 sequence, and/or partial R1 sequence.

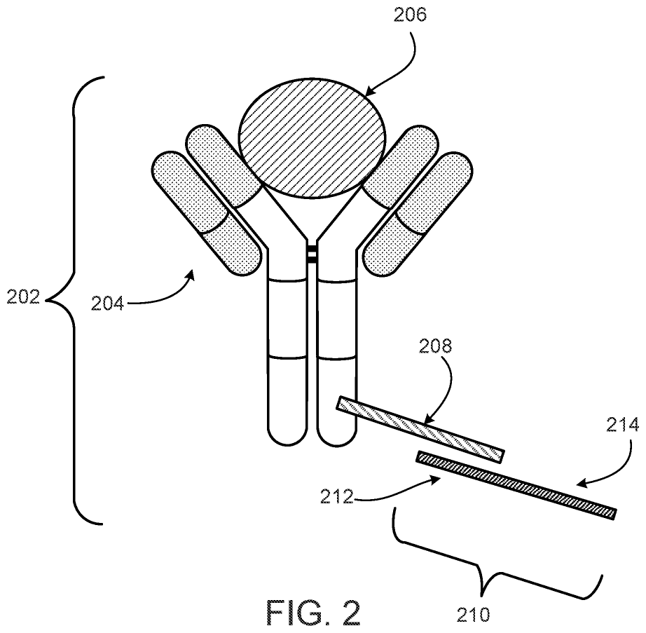

FIG. 2 is a schematic diagram of an exemplary antigen-binding complex 202 comprised of an analyte binding moiety 204 and an analyte binding moiety barcode 208. An analyte binding moiety 204 is a molecule capable of binding to an analyte 206. The analyte binding moiety can bind to the analyte 206 with high affinity and/or with high specificity. The antigen-binding moiety can include an analyte binding moiety barcode 208, a nucleotide sequence (e.g., an oligonucleotide), which can hybridize to at least a portion or an entirety of a capture oligonucleotide 210. The analyte binding moiety 204 can include a polypeptide and/or an aptamer (e.g., an oligonucleotide or peptide molecule that binds to a specific target analyte). The analyte binding moiety 204 can include an antibody or antibody fragment (e.g., an antigen-binding fragment). FIG. 2 additionally shows an exemplary capture oligonucleotide 210. A capture oligonucleotide, as previously described, comprises a sequence 212 that is substantially complementary to a least a portion of an oligonucleotide conjugated to an antigen-binding complex such as an analyte-binding moiety barcode 208. The capture oligonucleotide also includes an analyte capture sequence 214, capable of binding (e.g., hybridizing) to a capture domain of a capture probe.

The capture domain of the first capture probe and the second capture probe can be designed to interact (e.g., hybridize) to the first analyte capture sequence and/or the second analyte capture sequence. For example, the first capture domain and the second capture domain can have identical sequences. In some embodiments, the first capture domain and the second capture domain include identical sequences, where the sequence is a homopolymeric sequence. In some embodiments, the homopolymeric sequence is a poly(T) sequence. In some embodiments, the first capture domain and the second capture domain have different sequences from each other.

In some embodiments, the method includes extending a capture probe (e.g., the first capture probe) using the capture oligonucleotide as a template. In some embodiments, the method includes extending the second capture probe using the first probe as a template. In some embodiments, the method includes extending the second capture probe using the ligation product as a template. In some embodiments, extending the first capture probe and extending the second capture probe is performed with a DNA polymerase (e.g., extending with any suitable DNA polymerase). In some embodiments, extending includes extending a free 3′ end of the first capture probe and/or extending a free 3′ end of the second capture probe. In some embodiments, extending includes extending a free 3′ end of the analyte binding moiety barcode (e.g., an analyte binding moiety barcode bound to a capture oligonucleotide also bound to a capture probe). In some embodiments, extending includes extending a free 3′ end of the capture oligonucleotide (e.g., a capture oligonucleotide bound to a capture probe).

In some embodiments, the first capture probe includes one or more functional domains, a unique molecular identifier, a cleavage domain, and combinations thereof. In some embodiments, the second capture probe comprises one or more functional domains, a unique molecular identifier, a cleavage domain, and combinations thereof.

The biological sample can be any of the biological samples described herein. For example, in some embodiments, the biological sample is a tissue sample. In some embodiments, the tissue sample is a fixed tissue sample. In some embodiments, the biological sample is a tissue section. In some embodiments, the fixed tissue sample is a fixed tissue section. In some embodiments, the fixed tissue section comprises a formalin-fixed paraffin-embedded, a paraformaldehyde-fixed, an acetone-fixed, or a methanol-fixed tissue section. In some embodiments, the tissue sample comprises a fresh tissue sample or a frozen tissue sample (e.g., a fresh, frozen tissue sample).

In other embodiments, the biological sample is a clinical sample (e.g., whole blood, blood-derived products, blood cells, cultured tissue, cultured cells, or a cell suspension). In some embodiments, the biological sample is an organoid, embryonic stem cells, pluripotent stem cells, or any combination thereof. Non-limiting examples of an organoid include a cerebral organoid, an intestinal organoid, a stomach organoid, a lingual organoid, a thyroid organoid, a thymic organoid, a testicular organoid, a hepatic organoid, a pancreatic organoid, an epithelial organoid, a lung organoid, a kidney organoid, a gastruloid, a cardiac organoid, a retinal organoid, or any combination thereof. In other example embodiments, the biological sample can include diseased cells, fetal cells, immune cells, cellular macromolecules, organelles, extracellular polynucleotides, or any combination thereof.

In some embodiments, the second analyte is a nucleic acid. Non-limiting examples of nucleic acid analytes include DNA analytes such as genomic DNA, methylated DNA, specific methylated DNA sequences, fragmented DNA, mitochondrial DNA, in situ synthesized PCR products, and viral DNA.

Non-limiting examples of nucleic acid analytes also include RNA analytes such as various types of coding and non-coding RNA. Examples of the different types of RNA analytes include messenger RNA (mRNA), ribosomal RNA (rRNA), transfer RNA (tRNA), microRNA (miRNA), and viral RNA. The RNA can be a transcript (e.g., present in a tissue section). The RNA can be small (e.g., less than 200 nucleic acid bases in length) or large (e.g., RNA greater than 200 nucleic acid bases in length). Small RNAs mainly include 5.8S ribosomal RNA (rRNA), 5S rRNA, transfer RNA (tRNA), microRNA (miRNA), small interfering RNA (siRNA), small nucleolar RNA (snoRNAs), Piwi-interacting RNA (piRNA), tRNA-derived small RNA (tsRNA), and small rDNA-derived RNA (srRNA). The RNA can be double-stranded RNA or single-stranded RNA. The RNA can be circular RNA. The RNA can be a bacterial rRNA (e.g., 16s rRNA or 23s rRNA). The RNA can be from an RNA virus, for example RNA viruses from Group III, IV or V of the Baltimore classification system. The RNA can be from a retrovirus, such as a virus from Group VI of the Baltimore classification system.

In some embodiments, the target nucleic acid can include at least 1, at least 2, at least 3, at least 4, at least 5, at least 6, at least 7, at least 8, at least 9, or at least 10 disease-causing mutations (e.g., cancer-causing mutations). In some embodiments, the nucleic acid includes a single nucleotide polymorphism, gene amplification, or chromosomal translocation, deletion or insertion.

In some embodiments, the biological sample can be stained and/or imaged using any of the techniques described herein or known in the art (e.g., the biological sample can be stained and/or imaged between steps (a) and (b)). In some embodiments, the staining includes optical labels as described herein, including, but not limited to, fluorescent (e.g., fluorophore), radioactive (e.g., radioisotope), chemiluminescent (e.g., a chemiluminescent compound), a bioluminescent compound, calorimetric, or colorimetric detectable labels. In some embodiments, the staining includes a fluorescent antibody directed to a target analyte (e.g., cell surface or intracellular proteins) in the biological sample. In some embodiments, the staining includes an immunohistochemistry stain directed to a target analyte (e.g., cell surface or intracellular proteins) in the biological sample. In some embodiments, the staining includes a chemical stain, such as hematoxylin and eosin (H&E) or periodic acid-schiff (PAS). In some embodiments, staining the biological sample comprises the use of a biological stain including, but not limited to, acridine orange, Bismarck brown, carmine, coomassie blue, cresyl violet, DAPI, eosin, ethidium bromide, acid fuchsine, hematoxylin, Hoechst stains, iodine, methyl green, methylene blue, neutral red, Nile blue, Nile red, osmium tetroxide, propidium iodide, rhodamine, safranin, or any combination thereof. In some embodiments, significant time (e.g., days, months, or years) can elapse between staining and/or imaging the biological sample.

In some embodiments, permeabilizing the biological sample comprises contacting the non-permeabilized biological sample with a permeabilization agent, wherein the permeabilization agent is selected from an organic solvent, a detergent, and an enzyme, or a combination thereof. For example, the permeabilization agent can be selected from the group consisting of: an endopeptidase, a protease sodium dodecyl sulfate (SDS), polyethylene glycol tert-octylphenyl ether, polysorbate 80, and polysorbate 20, N-lauroylsarcosine sodium salt solution, saponin, Triton X-100™, and Tween-20™. In some embodiments, the endopeptidase is pepsin or proteinase K.

Templated Ligation

In some embodiments of methods provided here, templated ligation (e.g., RNA or DNA templated ligation) is used to interrogate spatial gene expression in a biological sample (e.g., a tissue section, a fixed tissue section). Templated ligation enables sensitive measurement of specific nucleic acids of interest that otherwise might be analyzed less sensitively with a whole transcriptomic approach. It provides advantages of compatibility with common histochemical stains and suitability for analysis of decade-old materials (e.g., FFPE samples) and exceedingly small microdissected tissue fragments.

In some embodiments, the steps of templated ligation include: (1) hybridization of pairs of probes (e.g., DNA, RNA, DNA/RNA probes) to nucleic acids (e.g., DNA or RNA) within a tissue section; (2) ligation of adjacently annealed probe pairs; (3) RNase (e.g., RNase H) treatment in the case of RNA-templated ligation that (i) releases the templated ligation products from the tissue (e.g., into solution) for downstream analysis and (ii) destroys unwanted DNA-templated ligation products; and optionally, (4) amplification of templated ligation products (e.g., by multiplex PCR).

In some embodiments, disclosed herein are methods of direct detection of RNA target-DNA probe duplexes without first converting RNA to cDNA by reverse transcription. In some aspects, RNA-templated ligation can include a DNA ligase. In some embodiments, RNA-templated ligation can include an RNA ligase. In some embodiments, RNA-templated ligation can include T4 RNA ligase.

In some embodiments, RNA-templated ligation is used for detection of RNA (e.g., mRNA), determination of RNA sequence identity, and/or expression monitoring and transcript analysis. In some embodiments, RNA-templated ligation allows for detection of a particular change in a nucleic acid (e.g., a mutation or single nucleotide polymorphism (SNP)), detection or expression of a particular nucleic acid, or detection or expression of a particular set of nucleic acids (e.g., in a similar cellular pathway or expressed in a particular pathology). In some embodiments, the methods that include RNA-templated ligation are used to analyze nucleic acids, e.g., by genotyping, quantitation of DNA copy number or RNA transcripts, localization of particular transcripts within samples, and the like. In some embodiments, systems and methods provided herein that include RNA-templated ligation identify single nucleotide polymorphisms (SNPs). In some embodiments, such systems and methods identify mutations.

In some embodiments, disclosed herein are methods of detecting RNA expression that include bringing into contact with a nucleic acid a first probe, a second probe, and a ligase (e.g., a T4 RNA ligase). In some embodiments, the first probe and the second probe are designed to hybridize to a target sequence such that the 5′ end of the first probe and the 3′ end of the second probe are adjacent and can be ligated, wherein at least the 5′-terminal nucleotide of the first probe and at least the 3′-terminal nucleotide of the second probe are deoxyribonucleotides (DNA), and wherein the target sequence comprises ribonucleotides (RNA). After hybridization, a ligase (e.g., T4 RNA ligase) ligates the first probe and the second probe if the target sequence is present in the target sample, but does not ligate the first probe and the second probe if the target sequence is not present in the target sample. The presence or absence of the target sequence in the biological sample can be determined by determining whether or not the first and second probes were ligated in the presence of a ligase. Any of a variety of methods can be used to determine whether or not the first and second probes were ligated in the presence of ligase, including but not limited to, sequencing the ligated product, hybridizing the ligated product with a detection probe that hybridizes only when the first and second probes were ligated in the presence of a ligase, restriction enzyme analysis, and other methods known in the art.

In some embodiments, two or more RNA analytes are analyzed using methods that include RNA-templated ligation. In some embodiments, when two or more analytes are analyzed, a first and second probe that are specific for (e.g., specifically hybridizes to) each RNA analyte are used.

In some embodiments, three or more probes are used in RNA-templated ligation methods provided herein. In some embodiments, the three or more probes are designed to hybridize to a target sequence such that the three or more probes hybridize adjacent to each other such that the 5′ and 3′ ends of adjacent probes can be ligated. In some embodiments, the presence or absence of the target sequence in the biological sample can be determined by determining whether or not the three or more probes were ligated in the presence of ligase.

In some aspects, the first probe is a DNA probe. In some aspects, the first probe is a chimeric DNA/RNA probe. In some aspects, the second probe is a DNA probe. In some aspects, the second probe is a chimeric DNA/RNA probe.

In some embodiments, reagents delivered to the biological sample are removed from the biological sample. In some embodiments, removing reagents includes washing the biological sample, for example, in a wash buffer. In some embodiments, after step (b) the method includes removing one or more unbound antigen-binding complexes. In some embodiments, the method includes after step (d) removing one or more unbound first probes. In some embodiments, the method includes removing one or more unbound second probes.

Direct Capture

Alternatively, RNA analytes (e.g., mRNA) can also be captured directly on an array (e.g., captured by a capture probe on the array) instead of an intermediate templated ligation product. For example, mRNA can be captured by a capture domain of a capture probe that binds (e.g., hybridizes) to the mRNA. In some embodiments, the capture domain of the capture probe includes a homopolymeric sequence. In some embodiments, the homopolymeric sequence is a poly(T) sequence. In some embodiments, the poly(T) sequence hybridizes to the poly(A) tail of an mRNA.

Direct capture of RNA analytes (e.g., mRNA) can also be multiplexed with the other methods described herein. For example, the antigen-binding complex and capture oligonucleotide methods described herein to detect analytes such as protein, can be performed in combination with the direct capture of mRNA as described above.

Blocking Probes

The methods provided herein can also utilize blocking probes to block the non-specific binding (e.g., hybridization) of the analyte capture sequence and the capture domain of a capture probe on an array. In some embodiments, following contact between the biological sample and the array, the biological sample is contacted with a plurality of antigen-binding complexes, where an antigen-binding complex includes an analyte capture sequence that is reversibly blocked with a blocking probe. In some embodiments, the analyte capture sequence is reversibly blocked with more than one blocking probe (e.g., 2, 3, 4, or more blocking probes). In some embodiments, the antigen-binding complex is blocked prior to binding the target analyte (e.g., a target protein).

In some embodiments, the analyte capture sequence of the capture oligonucleotide is blocked with one blocking probe. In some embodiments, the analyte capture sequence of the capture oligonucleotide is blocked with two blocking probes. In some embodiments, the analyte capture sequence of the capture oligonucleotide is blocked with more than two blocking probes (e.g., 3, 4, 5, or more blocking probes). In some embodiments, a blocking probe is used to block the free 3′ end of the analyte capture sequence of the capture oligonucleotide. In some embodiments, a blocking probe is used to block the 5′ end of the analyte capture sequence of the capture oligonucleotide. In some embodiments, two blocking probes are used to block both 5′ and 3′ ends of the analyte capture sequence of the capture oligonucleotide. In some embodiments, both the analyte capture sequence of the capture oligonucleotide and the capture probe domain are blocked.

In some embodiments, the blocking probes can differ in length and/or complexity. In some embodiments, the blocking probe can include a nucleotide sequence of about 8 to about 24 nucleotides in length (e.g., about 8 to about 22, about 8 to about 20, about 8 to about 18, about 8 to about 16, about 8 to about 14, about 8 to about 12, about 8 to about 10, about 10 to about 24, about 10 to about 22, about 10 to about 20, about 10 to about 18, about 10 to about 16, about 10 to about 14, about 10 to about 12, about 12 to about 24, about 12 to about 22, about 12 to about 20, about 12 to about 18, about 12 to about 16, about 12 to about 14, about 14 to about 24, about 14 to about 22, about 14 to about 20, about 14 to about 18, about 14 to about 16, about 16 to about 24, about 16 to about 22, about 16 to about 20, about 16 to about 18, about 18 to about 24, about 18 to about 22, about 18 to about 20, about 20 to about 24, about 20 to about 22, or about 22 to about 24 nucleotides in length).

In some embodiments, the blocking probe is removed prior to hybridizing the analyte capture sequence of the capture oligonucleotide to the first capture domain. For example, once the blocking probe is released from the analyte capture sequence of the capture oligonucleotide, the analyte capture sequence can bind to the first capture domain on the array. In some embodiments, blocking the analyte capture sequence of the capture oligonucleotide reduces non-specific background staining. In some embodiments, blocking the analyte capture sequence of the capture oligonucleotide allows for control over when to allow the binding of the analyte capture sequence of the capture oligonucleotide to the capture domain of a capture probe during a spatial workflow, thereby controlling the time of capture of the analyte capture sequence of the capture oligonucleotide on the array. In some embodiments, the blocking probes are reversibly bound, such that the blocking probes can be removed from the analyte capture sequence of the capture oligonucleotide during or after the time that antigen-binding complexes are in contact with the biological sample. In some embodiments, the blocking probes can be removed with RNAse treatment (e.g., RNAse H treatment). In some embodiments, the blocking probes are removed by increasing the temperature (e.g., heating) of the biological sample. In some embodiments, the blocking probes are removed enzymatically (e.g., cleaved). In some embodiments, the blocking probes are removed by a USER enzyme. In some embodiments, the blocking probes are removed by an endonuclease. In some embodiments, the endonuclease is endonuclease IV. In some embodiments, the endonuclease is endonuclease V.

In some embodiments, the determining in steps (e) and (f) include extension of the captured ligation products where the extension products comprise a complement of the templated ligation product and extension of the capture probe, extension of the capture oligonucleotide of the antigen-binding complex and/or extension of the analyte binding moiety barcode, releasing the extension products, or a complement thereof, from the spatial array, producing a library from the released extension products or complements thereof, and d) sequencing the library.

In some embodiments, determining includes extension of the capture probe using the directly captured RNA (e.g., mRNA) as a template, where the extension product comprises a complement of the RNA analyte (e.g., mRNA) and extension of the capture probe, extension of the capture oligonucleotide of the antigen-binding complexes, and/or extension of the analyte binding moiety barcode, releasing the extension products, or a complement thereof, from the spatial array, producing a library from the released extension products, or complements thereof, and sequencing the library.

### Analyte Transfer Configurations

Some steps of the methods described herein can be performed in a biological sample (e.g., in situ) prior to contacting the biological sample with the array including a plurality of capture probes. In some embodiments, the biological sample is disposed or placed on the array including the plurality of capture probes in step (a). In some embodiments, the biological sample is not disposed or placed on the array. For example, the biological sample can be placed or is located on a substrate (e.g., a slide) that does not include a spatial array. In some embodiments, the substrate including the biological sample can be aligned with the array (e.g., “sandwiched”) such that at least a portion of the biological sample is aligned with at least a portion of the array, transferring the analytes from the biological sample on the slide that is aligned with the arrayed slide to the arrayed slide.

In some embodiments, one or more products generated from the biological sample are released from the biological sample and migrate to a substrate comprising an array of capture probes where the products hybridize to the capture probes of the array. For example, the capture oligonucleotides that hybridize to the analyte binding moiety barcode, or a portion thereof, includes a first analyte capture sequence that hybridizes to a capture domain of a capture probe (e.g., a first capture probe). Similarly, the first probe that hybridizes to a second analyte (e.g., a nucleic acid analyte) includes a second analyte capture sequence that hybridizes to a capture domain of a capture probe (e.g., a second capture probe). In some embodiments, a ligation product is generated. For example, in embodiments where a first probe and a second probe are contacted with the biological sample the first probe and the second probe can both hybridize to the second analyte and be ligated together, thereby forming a ligation product. The ligation product includes the second analyte capture sequence from the first probe which can hybridize to a capture domain of a capture probe.

In some embodiments, the release and migration of the products to the substrate comprising the array of capture probes occurs in a manner that preserves the original spatial context of the products in the biological sample. In some embodiments, the biological sample is mounted on a first substrate and the substrate comprising the array of capture probes is a second substrate. In some embodiments, the method is facilitated by a sandwiching process. Sandwiching processes are described in, e.g., U.S. Patent Application Pub. No. 20210189475, WO 2021/252747, and WO2022/061152A. In some embodiments, the sandwiching process may be facilitated by a device, sample holder, sample handling apparatus, or system described in, e.g., U.S. Patent Application Pub. No. 20210189475, WO2021/252747, or WO2022/061152A.

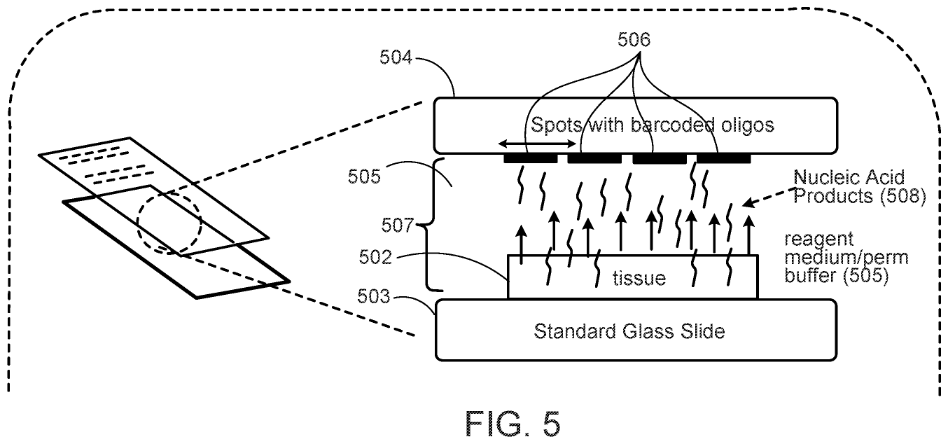

FIG. 5 is a schematic diagram depicting an exemplary sandwiching process 504 between a first substrate comprising a biological sample (e.g., a tissue section 502 on a slide 503) and a second substrate comprising a spatially barcoded array, e.g., a slide 504 that is populated with spatially-barcoded capture probes 506. During the exemplary sandwiching process, the first substrate is aligned with the second substrate, such that at least a portion of the biological sample is aligned with at least a portion of the array (e.g., aligned in a sandwich configuration). As shown, the second substrate (e.g., slide 504) is in a superior position to the first substrate (e.g., slide 503). In some embodiments, the first substrate (e.g., slide 503) may be positioned superior to the second substrate (e.g., slide 504). In some embodiments, the second substrate (e.g., slide 504) may be positioned superior to the first substrate (e.g., slide 503). In such embodiments, analytes and/or proxies thereof, would actively migrate against gravity. In some embodiments, active migration includes the use of electrophoresis. A reagent medium 505 (e.g., permeabilization solution) within a gap 507 between the first substrate (e.g., slide 503) and the second substrate (e.g., slide 504) creates a permeabilization buffer which permeabilizes or digests the biological sample 502 and the products described above 508 generated in the biological sample 502 may release, actively or passively migrate (e.g., diffuse) across the gap 507 toward the capture probes 506, and hybridize to the capture probes 506.

After the products described above 508 hybridize to the capture probes 506, an extension reaction may occur, thereby generating a spatially barcoded library. A polymerase can be used to generate a sequencing library associated with a particular spatial barcode. Barcoded libraries can be mapped back to a specific spot on a capture area of the capture probes 606. This data can be subsequently layered over a high-resolution microscope image of the biological sample, making it possible to visualize the data within the morphology of the tissue in a spatially-resolved manner. In some embodiments, the extension reaction can be performed separately from the sample handling apparatus described herein that is configured to perform the exemplary sandwiching process 504. The sandwich configuration of the sample 502, the first substrate (e.g., slide 503) and the second substrate (e.g., slide 504) can provide advantages over other methods of spatial analysis and/or analyte capture or proxies thereof (e.g., extension product(s)). For example, the sandwich configuration can reduce a burden of users to develop in house tissue sectioning and/or tissue mounting expertise. Further, the sandwich configuration can decouple sample preparation/tissue imaging from the barcoded array (e.g., spatially-barcoded capture probes 506) and enable selection of a particular region of interest of analysis (e.g., for a tissue section larger than the barcoded array). The sandwich configuration also beneficially enables spatial analysis without having to place a biological sample (e.g., tissue section) 502 directly on the second substrate (e.g., slide 504).

In some embodiments, the sandwiching process comprises: mounting the first substrate on a first member of a support device, the first member configured to retain the first substrate; mounting the second substrate on a second member of the support device, the second member configured to retain the second substrate, applying a reagent medium to the first substrate and/or the second substrate, the reagent medium comprising a permeabilization agent, operating an alignment mechanism (also referred to herein as an adjustment mechanism) of the support device to move the first member and/or the second member such that a portion of the biological sample is aligned (e.g., vertically aligned) with a portion of the array of capture probes and within a threshold distance of the array of capture probes, and such that the portion of the biological sample and the capture probe contact the reagent medium, wherein the permeabilization agent releases the extension product(s) from the biological sample.

The sandwiching process methods described above can be implemented using a variety of hardware components. For example, the sandwiching process methods can be implemented using a sample holder (also referred to herein as a support device, a sample handling apparatus, and an array alignment device). In some embodiments of a sample holder, the sample holder can include a first member including a first retaining mechanism configured to retain a first substrate comprising a sample. The first retaining mechanism can be configured to retain the first substrate disposed in a first plane. The sample holder can further include a second member including a second retaining mechanism configured to retain a second substrate disposed in a second plane. The sample holder can further include an alignment mechanism connected to one or both of the first member and the second member. The alignment mechanism can be configured to align the first and second members along the first plane and/or the second plane such that the sample contacts at least a portion of the reagent medium when the first and second members are aligned and within a threshold distance along an axis orthogonal to the second plane. The adjustment mechanism may be configured to move the second member along the axis orthogonal to the second plane and/or move the first member along an axis orthogonal to the first plane.

In some embodiments, the adjustment mechanism includes a linear actuator. In some embodiments, the linear actuator is configured to move the second member along an axis orthogonal to a to the plane or the first member and/or the second member. In some embodiments, the linear actuator is configured to move the first member along an axis orthogonal to the plane of the first member and/or the second member. In some embodiments, the linear actuator is configured to move the first member, the second member, or both the first member and the second member at a velocity of at least 0.1 mm/sec. In some embodiments, the linear actuator is configured to move the first member, the second member, or both the first member and the second member with an amount of force of at least 0.1 lbs.

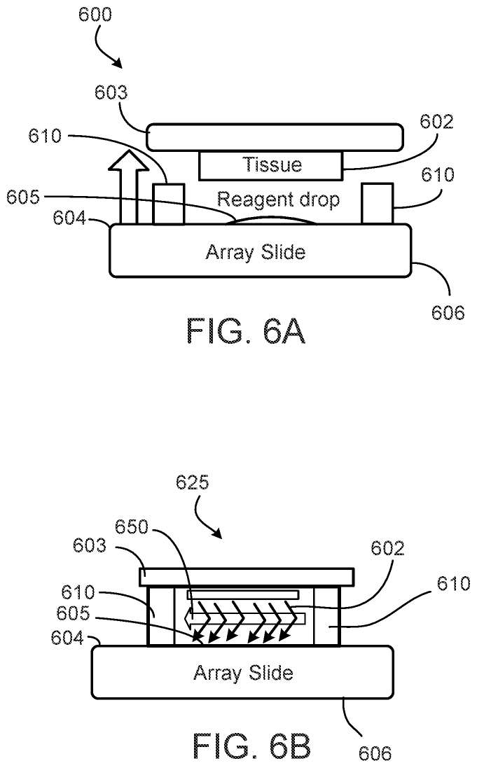

FIG. 6A shows an exemplary sandwiching process 600 where a first substrate (e.g., slide 603), including a biological sample 602 (e.g., a tissue section), and a second substrate (e.g., slide 604 including spatially barcoded capture probes 606) are brought into proximity with one another. As shown in FIG. 6A a liquid reagent drop (e.g., permeabilization solution 605) is introduced on the second substrate in proximity to the capture probes 606 and in between the biological sample 602 and the second substrate (e.g., slide 604 including spatially barcoded capture probes 606). The permeabilization solution 605 can release various products as described above that can be captured (e.g., hybridized) by the capture probes 606 of the array. As further shown, one or more spacers 610 can be positioned between the first substrate (e.g., slide 603) and the second substrate (e.g., slide 604 including spatially barcoded capture probes 606). The one or more spacers 610 may be configured to maintain a separation distance between the first substrate and the second substrate. While the one or more spacers 610 is shown as disposed on the second substrate, the spacer may additionally or alternatively be disposed on the first substrate.

FIG. 6B shows a fully formed sandwich configuration creating a chamber 650 formed from the one or more spacers 610, the first substrate (e.g., the slide 603), and the second substrate (e.g., the slide 604 including spatially barcoded capture probes 606) in accordance with some example implementations. In FIG. 6B, the liquid reagent (e.g., the permeabilization solution 605) fills the volume of the chamber 650 and can create a permeabilization buffer that allows products as described above to diffuse from the biological sample 602 toward the capture probes 606 of the second substrate (e.g., slide 604). In some aspects, flow of the permeabilization buffer may deflect products from the biological sample 602 and can affect diffusive transfer of products for spatial analysis. A partially or fully sealed chamber 650 resulting from the one or more spacers 610, the first substrate, and the second substrate can reduce or prevent flow from undesirable convective movement of transcripts and/or molecules over the diffusive transfer from the biological sample 602 to the capture probes 606.

### Kits

Also provided herein are kits including (a) an array comprising a plurality of capture probes comprising a first capture probe comprising: (i) a first spatial barcode and (ii) a first capture domain that binds a first analyte capture sequence, and a second capture probe of the plurality of capture probes comprising: (i) a second spatial barcode, and (ii) a second capture domain that binds a second analyte capture sequence; (b) a plurality of antigen-binding complexes, wherein an antigen-binding complex of the plurality of antigen-binding complexes comprises: (i) an antigen-binding moiety that binds a first analyte, and (ii) an antigen-binding moiety barcode, wherein the antigen-binding moiety barcode identifies a first antigen-binding complex; (c) a plurality of capture oligonucleotides, wherein a capture oligonucleotide of the plurality of capture oligonucleotides comprises: (i) a sequence substantially complementary to the analyte-binding moiety barcode, or a portion thereof, and (ii) the first analyte capture sequence; and (d) a plurality of first probes, wherein a first probe of the plurality of first probes comprises a sequence substantially complementary to a sequence of a second analyte, and wherein the first probe comprises a second analyte capture sequence that binds the second capture domain of the second capture probe.

In some embodiments, the kit includes a plurality of second probes, wherein a second probe comprises a sequence substantially complementary to the sequence of the second analyte, and wherein the first probe and the second probe are ligated.

In some embodiments, the kit includes a ligase.

In some embodiments, the first analyte is a protein. In some embodiments, the protein in an intracellular protein. In some embodiments, the protein is an extracellular protein. In some embodiments, the second analyte includes a nucleic acid. In some embodiments, the nucleic acid is DNA. In some embodiments, the DNA is genomic DNA. In some embodiments, the nucleic acid is RNA. In some embodiments, the RNA is mRNA.

In some embodiments, the antigen-binding complex comprises an antibody or antigen-binding fragment thereof.

In some embodiments, the kit includes one or more permeabilization reagents. In some embodiments, the one or more permeabilization reagents comprises an RNase, a DNase, a protease, a lipase, and combinations thereof.

In some embodiments, the first capture domain and the second capture domain comprise identical sequences. In some embodiments, the first capture domain and the second capture domain include a poly(T) sequence. In some embodiments, the first capture domain and the second capture domain include different sequences. In some embodiments, the first or second capture domain includes a poly (T) sequence while the other capture domain is a fixed, semi-fixed or random sequence. In some embodiments, the first and second capture domains are both fixed, semi-fixed, or random sequences. In some embodiments, the fixed sequences are different between the first and second capture domains, whereas in other embodiments the fixed sequences are the same between the first and second capture domains.

In some embodiments, the first spatial barcode and second spatial barcode comprise identical sequences. In some embodiments, the first spatial barcode and the second spatial barcode comprise different sequences.

In some embodiments, the first capture probe, the second capture probe, or both, comprises one or more functional domains, a unique molecule identifier, a cleavage domain, and combinations thereof.

In some embodiments, the kit includes one or more DNA polymerases.

### Compositions

Also provided herein are compositions comprising: an antigen-binding complex comprising (i) an analyte binding moiety and (ii) a conjugated oligonucleotide, wherein the conjugated oligonucleotide comprises an analyte binding moiety barcode; and a capture oligonucleotide bound to the analyte binding moiety barcode, or a portion thereof, wherein the capture oligonucleotide comprises (i) a sequence substantially complementary to the analyte binding moiety barcode and (ii) a first analyte capture sequence.

In some embodiments, the composition includes a plurality of first probes bound to a target analyte, wherein a probe of the plurality of first probes comprises (i) a sequence substantially complementary to a target analyte, or a portion thereof, and (ii) a second analyte capture sequence.

Also provided herein are compositions including: a) an array including a plurality of capture probes, where a capture probe comprises (i) a spatial barcode and (ii) a capture domain; b) a biological sample disposed on the array; c) a plurality of antigen-binding complexes, where an antigen-binding complex of the plurality of antigen-binding complexes includes: (i) an antigen-binding moiety that binds a first analyte, and (ii) an oligonucleotide including an analyte-binding moiety barcode, where the analyte-binding moiety barcode identifies the antigen-binding complex, and the antigen-binding complex is bound to the first analyte; d) a plurality of capture oligonucleotides, where a capture oligonucleotide of the plurality of capture oligonucleotides includes (i) a sequence substantially complementary to the analyte-binding moiety barcode, or a portion thereof, and (ii) a first analyte capture sequence, where the capture oligonucleotide is bound to analyte-binding moiety barcode, or a portion thereof; and e) a plurality of first probes, where a first probe of the plurality of first probes includes (i) a sequence substantially complementary to a sequence of a second analyte and (ii) a second analyte capture sequence that binds a second capture domain of a second capture probe, and wherein the first probe is bound to the second analyte.

Also provided herein are compositions including: a) an array including a plurality of capture probes, where a capture probe comprises (i) a spatial barcode and (ii) a capture domain; b) a biological sample disposed on a substrate; c) a plurality of antigen-binding complexes, where an antigen-binding complex of the plurality of antigen-binding complexes includes: (i) an antigen-binding moiety that binds a first analyte, and (ii) an oligonucleotide including an analyte-binding moiety barcode, where the analyte-binding moiety barcode identifies the antigen-binding complex, and the antigen-binding complex is bound to the first analyte; d) a plurality of capture oligonucleotides, where a capture oligonucleotide of the plurality of capture oligonucleotides includes (i) a sequence substantially complementary to the analyte-binding moiety barcode, or a portion thereof, and (ii) a first analyte capture sequence, where the capture oligonucleotide is bound to analyte-binding moiety barcode, or a portion thereof; and e) a plurality of first probes, where a first probe of the plurality of first probes includes (i) a sequence substantially complementary to a sequence of a second analyte and (ii) a second analyte capture sequence that binds a second capture domain of a second capture probe, and wherein the first probe is bound to the second analyte. In some embodiments, the biological sample is aligned with the array (e.g., sandwiched) and analytes are transferred from the biological sample to the array.

Also provided herein are compositions including a) an array including a plurality of capture probes, where a capture probe comprises (i) a spatial barcode and (ii) a capture domain; b) a biological sample disposed on the array; c) a plurality of antigen-binding complexes, where an antigen-binding complex of the plurality of antigen-binding complexes includes (i) an antigen-binding moiety that binds a first analyte, and (ii) an oligonucleotide comprising an analyte-binding moiety barcode, where the analyte-binding moiety barcode identifies the antigen binding complex, and the antigen-binding complex is bound to the first analyte; and d) a plurality of first probes, where a first probe of the plurality of first probes comprises (i) a sequence substantially complementary to a sequence of a second analyte and (ii) a second analyte capture sequence that binds a second capture domain of a second capture probe, and where the first probe is bound to the second analyte.

Also provided herein are compositions including a) an array including a plurality of capture probes, where a capture probe comprises (i) a spatial barcode and (ii) a capture domain; b) a biological sample disposed on a substrate; c) a plurality of antigen-binding complexes, where an antigen-binding complex of the plurality of antigen-binding complexes includes (i) an antigen-binding moiety that binds a first analyte, and (ii) an oligonucleotide comprising an analyte-binding moiety barcode, where the analyte-binding moiety barcode identifies the antigen binding complex, and the antigen-binding complex is bound to the first analyte; and d) a plurality of first probes, where a first probe of the plurality of first probes comprises (i) a sequence substantially complementary to a sequence of a second analyte and (ii) a second analyte capture sequence that binds a second capture domain of a second capture probe, and where the first probe is bound to the second analyte. In some embodiments, the biological sample is aligned with the array (e.g., sandwiched) and analytes are transferred from the biological sample to the array.

### EXAMPLES

### Example 1. Multiplex Detection of Analytes in a Biological Sample

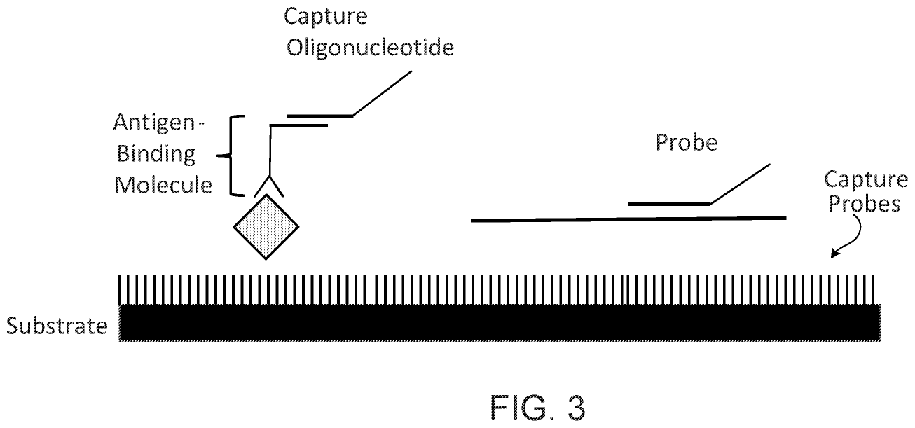

FIG. 3 shows an exemplary method for multiplex spatial capture of analytes in a biological sample. For example, an antigen-binding complex (e.g., an antibody, an antigen-binding fragment, etc.) interacts (e.g., binds) with an analyte (e.g., a first analyte), such as a protein target. The antigen-binding complex binds the analyte under experimental conditions. The antigen-binding complex includes an attached (e.g., conjugated) oligonucleotide that includes an analyte-binding moiety barcode. The analyte-binding moiety barcode identifies the antigen-binding complex.

A plurality of capture oligonucleotides are contacted with the biological sample under experimental conditions, where the capture oligonucleotide includes a sequence complementary to the oligonucleotide conjugated to the antigen-binding complex, or a portion thereof. In some examples, the capture oligonucleotide is substantially complementary to the analyte-binding moiety barcode, or a portion thereof. The capture oligonucleotide can also include a first analyte capture sequence. The first analyte capture sequence is capable of interacting with (e.g., hybridizing) to a capture domain (e.g., a first capture domain) of a capture probe on the substrate. A plurality of first probes are contacted with the biological sample, where the plurality of first probes are substantially complementary to a target analyte (e.g., nucleic acid) in the biological sample. It will be appreciated the plurality of capture oligonucleotides can be delivered to the biological sample before, after, or at about the same time as the plurality of first probes. A first probe can include a sequence substantially complementary to a target nucleic acid (e.g., mRNA) and a second analyte capture sequence (e.g., a sequence substantially complementary to a capture domain of a capture probe (e.g., a second capture probe)).

In some examples, a plurality of first probes and a plurality of second probes are delivered to the biological sample. For example, a first probe and a second probe can be complementary to a target analyte, such as an mRNA. The first probe and second probe each have sequences complementary to the target analyte and can be ligated to form a ligation product (e.g., a first ligation product) while hybridized to the nucleic acid. In such examples, either the first probe or the second probe can include a second analyte capture sequence (e.g., a sequence substantially complementary to a capture domain of a capture probe (e.g., a second capture probe)) on the substrate.

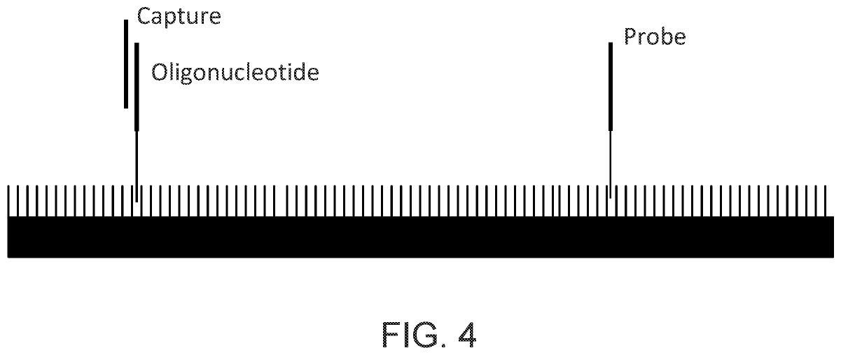

FIG. 4 shows capture of the capture oligonucleotide (e.g., capture oligonucleotide previously hybridized to the oligonucleotide conjugated to the antigen-binding complex) and the probe (e.g., the probe previously hybridized to the target analyte (e.g., mRNA)). FIG. 4 shows both the capture oligonucleotide and the probe interacting with a first capture probe and a second capture probe, respectively, on the substrate via their analyte capture sequences. FIG. 4 also shows the capture oligonucleotide released from the antigen-binding complex. The conjugated oligonucleotide portion of the antigen-binding complex can be released by various methods, such as, for example cleavage. The cleavage can be enzymatic cleavage or chemical cleavage. The probe or the ligation product can be released from the target nucleic acid by denaturation or treatment with an RNase.

After hybridizing the capture sequences to the associated capture domains of the capture probes, extension reactions including extending the first capture probe using the capture oligonucleotide as a template, extending the analyte binding moiety barcode, and/or extending the capture oligonucleotide using the capture probe as a template and extending the second capture probe by using the ligation product as an template can be performed. Alternatively, extension can also include extending the capture probe where RNA (e.g., mRNA) is directly capture and extending the capture probe using the mRNA as a template. Extension can include polymerization and/or reverse transcription, resulting in cDNA. In the case of the capture oligonucleotide the generated cDNA includes the analyte-binding moiety barcode sequence and in the case of the probe (e.g., ligation product) the generated cDNA includes a sequence of the target analyte or if directly captured the cDNA includes a complementary sequence of the analyte (e.g., mRNA). Optionally, the cDNA and/or a complement thereof can be amplified prior to downstream processing applications, such as creation of nucleic acid libraries and sequencing applications.

What is claimed is:

1. A method of determining a location of a first analyte and a location of a second analyte in a biological sample, the method comprising:
a) providing an array comprising a plurality of capture probes, wherein a first capture probe of the plurality of capture probes comprises: (i) a first spatial barcode, and (ii) a first capture domain that hybridizes to a first analyte capture sequence;
b) contacting the biological sample with
(i) a plurality of antigen-binding complexes, wherein an antigen-binding complex of the plurality of antigen-binding complexes comprises: (1) an antigen-binding moiety that binds a first analyte, and (2) an oligonucleotide comprising an analyte-binding moiety barcode, wherein the analyte-binding moiety barcode identifies the antigen-binding complex;
(ii) a plurality of capture oligonucleotides, wherein a capture oligonucleotide of the plurality of capture oligonucleotides comprises: (1) a sequence substantially complementary to the analyte-binding moiety barcode, or a portion thereof, and (2) the first analyte capture sequence that hybridizes to the first capture domain of the first capture probe;
(iii) a plurality of first probes, wherein a first probe of the plurality of first probes comprises a sequence substantially complementary to a sequence of a second analyte and hybridizes to the second analyte, and wherein the first probe comprises a second analyte capture sequence that hybridizes to a second capture domain of a second capture probe;
c) determining (i) a sequence of the first spatial barcode, or a complement thereof, and (ii) the sequence of the analyte-binding moiety barcode, or a complement thereof, and correlating the determined sequences of (i) and (ii) to a location of the first analyte in the biological sample; and
d) determining (i) a sequence of the second spatial barcode, or a complement thereof, and (ii) the sequence of the first probe, or a complement thereof, and correlating the determined sequences of (i) and (ii) to a location of the second analyte in the biological sample.

2. The method of claim 1, wherein the method further comprises contacting a plurality of second probes with the biological sample, wherein the first probe and a second probe of the plurality of second probes each comprise one or more sequences substantially complementary to sequences of the second analyte and wherein the second probe hybridizes to the second analyte; and
ligating the first probe and the second probe with a ligase, thereby generating a ligation product.

3. The method of claim 2, wherein the method further comprises releasing the ligation product from the second analyte comprising the use of an RNase or heat.

4. The method of claim 2, wherein the ligation product comprising the second analyte capture sequence hybridizes to a second capture domain of a second capture probe, and wherein the second capture probe comprises a second spatial barcode.

5. The method of claim 2, wherein the method further comprises: determining (i) a sequence of a second spatial barcode, or a complement thereof, and (ii) the sequence of the ligation product, or a complement thereof, and correlating the determined sequences of (i) and (ii) to a location of the second analyte in the biological sample.

6. The method of claim 1, wherein the first antigen-binding complex is an antibody, or an antigen-binding fragment thereof, and the first analyte comprises a protein and the second analyte comprises a nucleic acid.

7. The method of claim 1, wherein after step (b) the method further comprises removing one or more unbound antigen-binding complexes and/or removing one or more unbound first probes or one or more unbound second probes.

8. The method of claim 1, wherein the first capture domain and the second capture domain comprise a poly(T) sequence or wherein the first capture domain and the second capture domain comprise different sequences.

9. The method claim 1, wherein the first capture probe and the second capture probe comprise: one or more functional domains, a unique molecule identifier, a cleavage domain, and combinations thereof.

10. The method of claim 1, wherein the method further comprises extending the first capture probe using the capture oligonucleotide as a template and/or extending the second capture probe as a template using the first probe as a template, wherein the extending comprises the use of a polymerase.

11. The method of claim 2, wherein the method further comprises extending the second capture probe using the ligation product as a template, wherein the extending comprises the use of a polymerase.

12. The method claim 1, wherein the method further comprises permeabilizing the biological sample.

13. The method of claim 1, wherein the method further comprises imaging the biological sample and/or staining the biological sample, wherein the staining comprises hematoxylin and eosin staining or immunofluorescence.

14. The method of claim 1, wherein the biological sample is a tissue sample or a tissue section, wherein the tissue section is a fresh-frozen tissue section or a fixed tissue section, wherein the fixed tissue section comprises a formalin-fixed paraffin-embedded tissue section, a paraformaldehyde-fixed tissue section, an acetone-fixed tissue section, or a methanol-fixed tissue section.

15. The method of claim 1, wherein the determining in step (e), step (f), or both comprises sequencing.

16. The method of claim 1, wherein the antigen-binding complex further comprises a linker, wherein the linker is disposed between the analyte-binding moiety and the analyte-binding moiety barcode wherein the linker is a cleavable linker, and wherein the cleavable linker is a photo-cleavable linker or an enzyme cleavable linker.

17. The method of claim 1, wherein the method further comprises blocking the first analyte capture sequence and/or second analyte capture sequence with one or more blocking probes.

18. The method of claim 1, wherein the biological sample is disposed on the array.

19. The method of claim 1, wherein the biological sample is disposed on a substrate.

20. The method of claim 19, wherein the method further comprises aligning the substrate with the array, such that at least a portion of the biological sample is aligned with at least a portion of the array.
